(19) United States
(12) Patent Application Publication
(10) Pub. No.: US 2021/0346444 A1
(43) Pub. Date: Nov. 11, 2021

(54) USE OF NELUMBO NUCIFERA LEAF EXTRACT IN PREPARING A PHARMACEUTICAL COMPOSITION FOR THE INCREASE OF ADULT HIPPOCAMPAL NEUROGENESIS

(71) Applicant: Chung Shan Medical University, Taichung City (TW)

(72) Inventors: Chau-Jong Wang, Taichung City (TW); Yen-Ling Chang, Taichung City (TW)

(73) Assignee: Chung Shan Medical University, Taichung City (TW)

(21) Appl. No.: 16/867,577

(22) Filed: May 6, 2020

(51) Int. Cl. A61K 36/185; A61K 9/00; A61P 25/28

(52) U.S. Cl. CPC A61K 36/185; A61P 25/28; A61K 9/0053

(57) ABSTRACT

The present invention provides a composition comprising Nelumbo nucifera leaf extract and a method for the increase of adult hippocampal neurogenesis.

### BACKGROUND OF THE INVENTION

### Field of the Invention

Nelumbo nucifera leaf extract in preparing a pharmaceutical composition for the increase of adult hippocampal neurogenesis.

### Description of Prior Art

The hippocampus is located inside the temporal lobe of the vertebrate brain and appears in pairs on the left and right hemispheres of the brain. The hippocampus consists of dentate gyrus (DG), cornu ammonis (CA1-4), subiculum, presubiculum, parasubiculum, entorhinal cortex (EC) and other regional composition. The hippocampus is mainly responsible for cognitive abilities such as learning, short-term memory, long-term memory, emotional behavior, and spatial positioning. Many studies have shown that memory will form short-term memory quickly in the hippocampus, and then gradually integrate into the cerebral cortex to form long-term memory and store it. The hippocampus plays a vital role in the formation of memory. If the number of nerve cells in the hippocampus can be maintained, there will be an opportunity to slow down the neurodegeneration caused by aging or trauma.

Dementia is a broad category of brain disease that causes long-term and gradual decrease in thinking and memory that is severe enough to affect daily functions. Other common symptoms include emotional problems, difficulties with language, and a decrease in motivation. Patients will experience cognitive dysfunction, including memory and learning function, attention, language function, perception, space, motor integration function, reasoning, calculation, organization and planning, and other executive dysfunction or social cognitive function. Dementia is mainly divided into dementia caused by neurodegenerative diseases, vascular dementia, and other secondary dementia. Most patients are degenerative dementia, including Alzheimer's disease, Frontotemporal dementia, dementia with Lewy bodies, Parkinson's disease, or Huntington's disease caused by dementia, etc. The other type is vascular dementia, which is mainly due to brain stroke and chronic cerebral vascular lesions, resulting in poor blood circulation in the brain, leading to brain cell death and mental retardation. In addition, there are other factors that cause dementia, which may have a chance to recover after treatment. The causes of this type of dementia include malnutrition, intracranial lesions, abnormal metabolism, central nervous system infection, poisoning, or other factors.

Neurodegenerative Diseases

Neurodegenerative disease is a slow pathological process involving genetic and non-genetic environmental factors. The structure and function of the brain will change with age. The most common neurodegenerative disease in the world is Alzheimer's disease and Parkinson's disease.

Alzheimer's disease is the most common and irreversible progressive neurodegenerative disease. Patients have symptoms such as memory loss, cognitive impairment, mood swings, loss of direction, and eventually delirium. There is still no effective treatment to date. The main pathological features of Alzheimer's disease are the appearance of β-amyloid plaques outside nerve cells and the appearance of neurofibrillary tangles in nerve cells. Beta-amyloid (Aβ) is derived from a larger protein molecule present in the membrane surrounding nerve cells. Aβ will aggregate to form small pieces of sticky protein, and gradually accumulate to form plaques. The small protein blocks can interfere with cell-to-cell synaptic signals, which can lead to over-excited neurons and abnormal neural networks, causing learning and memory damage. Neurofibrillary tangles are mainly caused by the excessive phosphorylation of Tau protein, which causes microtubules distortion, and then accumulate in cells. They can damage the neuron transport system, lead to progressive neurodegeneration, and cause dementia. In addition to β-amyloid plaques and neurofibrillary tangles, the main symptoms of Alzheimer's disease are also accompanied by neuropil threads, dystrophic neurites, and NF-κB activation in astrocytes and microglia leads to loss of nerve cell function and death. These symptoms usually start from the heterogeneous cortical area of the medial temporal lobe, that is, the location of the entorhinal cortex and the hippocampus. In turn, it affects other cortical areas connected to it, so it has relatively little effect on the main sensory area, motor area, and visual area.

Parkinson's disease is the second most common progressive neurodegenerative disease in the world, and it occurs in the elderly over 60 years old. Patients will experience symptoms such as tremor, muscular rigidity, bradykinesia, postural abnormality, fatigue, hyposmia depression, and cognitive problems. The main symptom of Parkinson's disease is the accumulation of Lewy bodies formed by the misfolding and accumulation of α-synuclein protein in the substantia nigra, leading to neuronal dysfunction. In addition, some studies have shown that post-translational modifications of α-synuclein, such as phosphorylation and ubiquitination, will accelerate the aggregation of α-synuclein protein and produce neurotoxicity leading to neuronal cell death. Dopamine neurons in the substantia nigra secrete dopamine and transfer it to the basal nucleus in the brain. When dopamine neurons degenerate, dopamine secretion is reduced, which is the main pathogenic factors of Parkinson's disease. Decreased dopamine secretion will cause insufficient dopamine delivered to the striatum that controls movement and balance, and will not be able to maintain balance with acetylcholine. Therefore, the symptoms of dyskinesia will eventually appear, and the symptoms of Parkinson's disease will only appear after 70 to 80% of apoptosis.

Obesity and Neurodegenerative Diseases

According to statistics of the World Health Organization (WHO) in 2016, about 2 billion people worldwide are overweight. Obesity can cause complications such as cardiovascular disease, type 2 diabetes, sleep apnea, and cancer, which seriously affect people's health. Previous studies have shown that obese people (BMI≥40) and diabetic patients have poor cognitive ability and a higher probability of suffering from dementia. Another study showed that the 6-week-old leptin-deficient mice have nearly double the body weight as controls, but their brain volume was 10% smaller than control animals of the same age. Furthermore, the expression of phosphohistone H3, which is the proliferation marker and doublecortin (DCX), which is the differentiation indicator of immature nerve cells are both lower in the hippocampus of the obese mice. Therefore, it can be realized that obesity will cause a significant reduction in neuronal proliferation and differentiation ability. However, in this study, it was shown that obesity does not directly affect memory, learning, and cognitive abilities. In the long run, when the ability of nerve proliferation and differentiation is reduced, it will indirectly increase the possibility of suffering from neurodegenerative diseases.

It has recently been shown that hypercholesterolemia is closely related to neurodegenerative diseases, and abnormal cholesterol metabolism is one of the important risk factors for Alzheimer's disease. Due to the mutation of the low-density lipoprotein receptor (LDLr) gene, the LDL in the blood cannot be metabolized by the liver, and the cholesterol in the blood is abnormally high. In addition to causing atherosclerosis and cardiovascular disease, it also destroys the proliferation of adult hippocampal gyrus nerve precursor cells and affects cognitive ability.

Adult Neurogenesis

Nerve cells are generated by the sequential differentiation of neural stem cells and neural progenitor cells. In the past, it was believed that nerve cell regeneration only occurred in the embryonic stage and infant stage, while adult individuals did not have nerve regeneration. However, in 1967, a neurobiologist, Joseph Altman discovered new neurons in the hippocampus of adult guinea pigs and proposed for the first time that the brain of adult mammals also has the idea of neural regeneration, but this result was caused at the time considerable controversy. In 1981, Dr. Fernando Nottebohm discovered that in the spring, the brain regions of mature male canaries that control singing, including Hyperstriatum ventral, pars caudal nucleus (HVc) and nucleus robustus archistriatalis (RA), have increased the number of synapses and nuclear regions. The size increased by 99% and 76% relative to the end of reproduction period. So it can be used to learn and produce new songs. However, when the reproduction is over, the number of neurons and the size of the brain area will decrease. Until the next spring, the male canary will produce new neurons and produce songs different from the previous year. In 1983, research showed that injecting testosterone into an adult female canary can induce neural regeneration and differentiation in its HVc brain area and increase its size by twice. And the female bird who would not sing would start singing like the male bird. It was discovered that adult canary brain neurons have the ability to self-rejuvenation, thus breaking the previous rule that once an animal becomes an adult, the brain will not change for a lifetime.

To date, many studies have confirmed that the two main sites of adult neural regeneration and differentiation are the subgranular zone (SGZ) of the hippocampal dentate gyrus and the subventricular zone (SVZ) of the lateral ventricle. Previous studies have found that the dentate gyrus of young adult rats (9-10 weeks old) can regenerate approximately 9000 cells a day and most of the cells will differentiate into granule neurons and exist for more than 8 months. In addition, the phenomenon of adult neural regeneration found in rodents, the results published by scientists such as Maura Boldrini in 2018 showed that the adult hippocampus continues to retain the ability of neural regeneration until old age (over 65 years old). 28 healthy human hippocampus tissues (age range from 14 to 79 years old) within 26 hours of death were collected and by using immunohistochemical staining and immunofluorescence assays to observe the expression and distribution of doublecortin (an immature nerve cell marker protein), NeuN (a mature neuron nuclear marker protein), SOX2, and Nestin (immature neural progenitor marker protein) in the hippocampus. The results show that the quiescent stem cell pool, angiogenesis, and neuroplasticity decrease with age, but the hippocampal dentate gyrus area and dentate gyrus neural progenitor cells, immature and the number of mature granule neurons, and glial has no significant change in any age group, thus confirming that the adult hippocampus still has nerve regeneration until old age.

Research has found that there are about 700 new neurons in the dentate gyrus of the human brain every day. However, new neurons do not expand the brain area indefinitely, because when there are too many brain cells, programmed cell death is initiated to maintain the self-renewal ability. At present, many scientists believe that some healthy elderly people do not decrease their cognitive ability or suffer from neuropsychiatric diseases as they age, mainly because their hippocampus retains the ability to regenerate nerves. Therefore, how to effectively increase adult neural regeneration and maintain its survival rate and function is an important issue to slow down neurodegenerative diseases.

Human Neuroblastoma, SH-SY5Y Cell Line

SH-SY5Y cell line was taken from metastatic bone tumor biopsy cell line (SK-N-SH) metastasized to bone cancer and was established by three clones (SK-N-SH→SH-SY→SH-SY5-→SH-SY5Y). This cell line has both suspension and attachment cell growth patterns and expresses tyrosine hydroxylase, dopamine beta-hydroxylase, and dopamine transporter. The characteristics of this cell line are similar to dopaminergic neurons, so it is often used in neuroscience research today.

Neurogenesis-Related Proteins and Signal Transmission Pathways

Doublecortin (DCX) is a microtubule-associated protein (MAP) that serves as a key protein in neuronal dispersion and cortex lamination during the development of the cerebral cortex. DCX is mainly expressed in the first two weeks of neuron generation and can be used to identify neural precursor cells and early migration and differentiation (immature neurons), so it is often used as an important indicator of neural regeneration.

Neuronal nuclei (NeuN), also known as RNA binding protein fox-1 homolog 3 (Rbfox3), is specifically expressed in the nucleus of neurons and is a kind of highly specific neuron with mitotic neurons. NeuN is mainly responsible for regulating RNA transcription and neuron splicing and participating in synapse generation and neuron differentiation, so it is often used to mark the nucleus of mature neurons or post-mitotic neurons. Some cells do not express NeuN, such as sympathetic ganglion cells, retinal photoreceptor cells, and dentate nucleus neurons.

Nestin is a neuroepithelial stem cell protein and belongs to the sixth type of intermediate filament protein. It is mainly expressed in neural stem cells during brain development. When the brain is mature, the expression of Nestin will drop sharply, and replaced by expression of neurofilament protein. In addition, Nestin is also expressed in many different tissues, stem cells or progenitor cells, such as islets, skeletal muscles, satellite cells, testes, hair follicles, heart and non-hematopoietic fractions of bone and other tissues. It is often used as a molecular marker for neuroepithelial stem cells or immature neural progenitor cells. In addition, studies have shown that the expression of Nestin in many malignant tumors is also very high, such as osteosarcoma, neuroblastoma, glioma, melanoma, pancreatic cancer, and prostate cancer and is related to the invasion of glioblastoma, angiogenesis of the tumor, and the spread of epithelial and non-epithelial cell tumors. So it is often regarded as a molecular marker of invasive phenotype.

Brain-Derived Neurotrophic Factor (BDNF)

BDNF is the most abundant neurotrophin in the central nervous system. Mature BDNF is formed by cutting the precursor BDNF (proBDNF) stored in axons or dendrites. The mature BDNF will bind to tropomyosin receptor kinase B (TrkB) and activate Ras/MAPK/ERK pathway, IRS-1/PI3K/AKT pathway, and PLC/DAG/IP3 pathway to ultimately increase the expression of phosphorylated CREB to regulate nerve growth, differentiation and remodeling. Previous studies have shown that the expression level of BDNF is significantly lower in neurodegenerative diseases such as Parkinson's disease, multiple sclerosis, and Huntington's disease. BDNF stimulates and regulates neural stem cells to generate new neurons to replace the lost neurons, so it plays a vital role in learning and memory. In addition, the study also found that the atrophy of the hippocampus will be accompanied by a decrease in the secretion of brain-derived neurotrophic factors and the occurrence of amnesia. Therefore, if it can effectively increase the secretion of BDNF in the brain of the patient, it can improve the neurodegenerative situation.

Nelumbo nucifera Leaf

Nelumbo nucifera, also known as Indian lotus, sacred lotus, bean of India, Egyptian bean or simply lotus, is one of two extant species of aquatic plant in the family Nelumbonaceae. The roots, stems, leaves, flowers, fruits and seeds of Nelumbo nucifera all contain alkaloids, flavonoids, glycosides and vitamins, which have the effects of antioxidant, anti-inflammatory and lipid-lowering. Nelumbo nucifera leaf contains alkaloids, flavonoids, vitamin C, tartaric acid, oxalic acid, succinic acid, triterpenes, glycosides, tannins and polyphenols. It has antioxidant, anti-inflammatory, anti-cancer, anti, antifungal, hypolipidemic and Multiple functions such as lowering blood sugar. Nelumbo nucifera leaf contains various alkaloids, among which nuciferine and N-nornuciferine are the two main alkaloids in lotus leaf. Many studies indicate that alkaloids can inhibit fat differentiation and pancreatic lipase. Alkaloids can also reduce melanin production by inhibiting the expression of tyrosinase and tyrosinase-related protein-2. In addition, alkaloids have multiple pharmacological activities such as anti-inflammatory, anti-oxidation, anti-cancer, anti-aging, lowering blood sugar and hypertension. In recent years, more studies have indicated that alkaloids can perform antioxidant, increase the concentration of γ-Aminobutyric acid (GABA), reduce the precipitation of β-amyloid and α-synuclein aggregates and inhibits the activities of monoamine oxidase and acetylcholinesterase to delay the progression of neurodegenerative diseases such as Alzheimer's disease, Parkinson's disease and Huntington's disease.

The Nelumbo nucifera leaf mainly contains flavonoids such as lotoside, polymeric proanthocyanidin, quercetin, isoquercitrin, etc. I It mainly has antioxidant functions and anti-inflammatory effects. In addition, it can reduce the discomfort symptoms such as foot palsy and pain caused by diabetic neuropathy. Furthermore, Nelumbo nucifera leaf contains gallic acid, caffeic acid and catechin, which has anti-obesity, anti-inflammatory, antioxidant and anti-cancer effects. Previous studies also indicated that Nelumbo nucifera leaf extract can effectively slow down alcoholic hepatitis and diabetes, and can reduce angiogenesis and metastasis of triple negative breast cancer by inhibiting the PI3K/AKT/ERK pathway.

Nowadays, the aging of the population has become an important issue that cannot be underestimated. With the increase in the average lifespan of human beings, the rate of people suffering from neurodegenerative diseases is increasing. The elderly population has increased significantly, and will tend to double with age. The US Centers for Disease Control and Prevention pointed out in 2016 that Alzheimer's disease has become the sixth leading cause of death among Americans, while Taiwan's statistics in 2019 found that the proportion of the elderly population over 65 years old accounted for 14.9% of the total population. The Taiwan Dementia Association estimates that by 154 years of the Republic of China, the number of people with dementia over the age of 65 in China will grow by 2 to 3 times today. In addition to the inconvenience in life of patients with neurodegenerative diseases, patients and their families have to bear huge economic and physical burdens for a long time. Therefore, how to reduce the incidence of neurodegenerative diseases has become a public issue that cannot be ignored worldwide.

### SUMMARY OF THE INVENTION

The present invention provides a method of increasing adult hippocampal neurogenesis in subject, comprising administering a composition comprising an effective amount of Nelumbo nucifera leaf extract to the subject, wherein the Nelumbo nucifera leaf extract is Nelumbo nucifera leaf water extract (NLWE), or Nelumbo nucifera leaf alcoholic extract (NLAE).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. DCX-positive neural progenitor cells were stained in the subgranular zone of the hippocampal dentate gyrus. Mice were fed with NLWE (10 g/kg) or NLAE (1 g/kg) for 14 days. A. Representative photomicrographs were taken at magnifications of 200×. The red arrows: DCX-positive cells. B. The intensity of the DCX-positive staining was quantified. The data are expressed as the means±SD (n=6) and statistically analyzed with a Student's t-test. *p<0.01, **p<0.001 compared with the control group.

FIG. 2. NeuN-positive neural progenitor cells were stained in the subgranular zone of the hippocampal dentate gyrus. Mice were fed with NLWE (10 g/kg) or NLAE (1 g/kg) for 14 days. A. Representative photomicrographs were taken at magnifications of 200×. The red arrows: NeuN-positive cells. B. The intensity of the NeuN-positive staining was quantified. The data are expressed as the means±SD (n=6) and statistically analyzed with a Student's t-test. * p<0.05, **p<0.01 compared with the control group.

FIG. 3. PCNA-positive neural progenitor cells were stained in the subgranular zone of the hippocampal dentate gyrus. Mice were fed with NLWE (10 g/kg) or NLAE (1 g/kg) for 14 days. A. Representative photomicrographs were taken at magnifications of 200×. The red arrows: PCNA-positive cells. B. The intensity of the PCNA-positive staining was quantified. The data are expressed as the means±SD (n=6) and statistically analyzed with a Student's t-test. * p<0.05, **p<0.01 compared with the control group.

FIG. 4. NLWE and NLAE increased BDNF levels in serum. Mice were fed with NLWE (10 g/kg) or NLAE (1 g/kg) for 14 days. BDNF levels in serum were determined by ELISA. The values were the means±SD (n=6). Results were statistically analyzed with a one-way ANOVA. *p<0.05 compared with the control group.

FIG. 5. NLWE and NLAE treatment increased doublecortin (DCX) levels in the mice hippocampus. Mice were fed with NLWE (10 g/kg) or NLAE (1 g/kg) for 14 days. Protein extracts from mice hippocampus were measured by western blotting to detect DCX. β-actin was used as a loading control. Data was presented as means±SD from three independent experiments and statistically analyzed with a one-way ANOVA. *p<0.05 compared with the control group.

FIG. 6. NLWE and NLAE treatment activated BDNF/TrkB/CREB signaling pathway to promote hippocampal neurogenesis. Mice were fed with NLWE (10 g/kg) or NLAE (1 g/kg) for 14 days. Protein extracts from mice hippocampus were measured by western blotting to detect BDNF, TrkB, and CREB. β-actin was used as a loading control. Data was presented as means±SD from three independent experiments and statistically analyzed with a one-way ANOVA. *p<0.05 compared with the control group.

FIG. 7. The cytotoxicity effects of NLAE in SH-SY5Y by MTT. SH-SY5Y cell (1×10⁴ cells/well) were incubated with different concentration of NLAE (0, 25, 50, 100 μg/mL) in 96 well plate for 24 hours. Cell viability were analyzed by MTT assay. The data was mean±SD for three replicates per treatment and statistically analyzed with a Student's t test. *p<0.05 compared with the control group.

FIG. 8. BrdU cell proliferation ELISA assay for assessment of proliferation activity in NLAE-treated SH-SY5Y cells. SH-SY5Y cell (1×10⁴ cells/well) were incubated with different concentration of NLAE (0, 25, 50, 100 μg/mL) in 96 well plate for 24 hours. Cells were assayed for proliferation by measuring BrdU incorporation during DNA synthesis in proliferating cells. The data was mean±SD for three replicates per treatment and statistically analyzed with a Student's t test. *p<0.05 compared with the control group (0 μg/mL NLAE).

FIG. 9. NLAE treatment increased doublecortin (DCX) level in SH-SY5Y cells. SH-SY5Y cell (2.37×10⁶ cells/flask) were incubated with different concentration of NLAE (0, 25, 50, 100 μg/mL) in 75T flask for 24 hours. Protein extracts from SH-SY5Y cell were measured by western blotting to detect DCX. β-actin was used as a loading control. Data was presented as means±SD from three independent experiments and statistically analyzed with a Student's t test. *p<0.05 compared with the control group (0 μg/mL NLAE).

### DETAILED DESCRIPTION OF THE INVENTION

The hippocampus plays a crucial role in the regulation of cognitive abilities. The subgranular zone of the dentate gyrus has the phenomenon of adult neural regeneration until old age, but the incidence of neural regeneration will decrease with age. In the past, studies have confirmed that the effects of promoting neural regeneration in the hippocampus of mice and increasing the proliferation and migration of neural stem cells are to improve neurodegenerative diseases.

The present invention provides a method of increasing adult hippocampal neurogenesis in the subject, comprising administering a composition comprising an effective amount of Nelumbo nucifera leaf extract to the subject, wherein the Nelumbo nucifera leaf extract is Nelumbo nucifera leaf water extract (NLWE), or Nelumbo nucifera leaf alcoholic extract (NLAE).

According, the invention provides a use of Nelumbo nucifera leaf extract in preparing a composition for the increase of adult hippocampal neurogenesis, wherein the Nelumbo nucifera leaf extract is Nelumbo nucifera leaf water extract (NLWE), or Nelumbo nucifera leaf alcoholic extract (NLAE).

According to one aspect of the invention, wherein the Nelumbo nucifera leaf extract composition is a pharmaceutical composition or a food composition.

In one embodiment, wherein the pharmaceutical composition further comprises a pharmaceutically acceptable carrier, adjuvant or excipient. In another embodiment, wherein the food composition is selected from the group consisting of food, beverages, health foods, and dietary supplements.

In an additional aspect of the invention, the Nelumbo nucifera leaf extract is a Nelumbo nucifera leaf water extract (NLWE). In one embodiment, the Nelumbo nucifera leaf water extract is obtained by grinding dried lotus leaves and then dissolving, filtering, centrifuging, and freeze-dried.

In an additional further aspect of the invention, the Nelumbo nucifera leaf extract is a Nelumbo nucifera leaf alcoholic extract (NLAE), wherein the Nelumbo nucifera leaf alcoholic extract (NLAE) is obtained by extracting the dried Nelumbo nucifera leaf water extract with an alcohol. In one embodiment, wherein the alcohol is methanol.

The term “prevention” as used herein refers to delaying the onset of symptoms or reducing the appearance of disease in subjects suffering from the disease, and “treatment” means relieving or improving the symptoms of susceptible subjects.

The term “subject” as used herein means an animal, especially a mammal. In a preferred embodiment, the term “subject” means “human”.

As used herein, the term “effective amount” means that the active ingredient is used alone or in combination with other drugs to show the effect of increasing adult hippocampal neurogenesis, and to reduce or prevent neurodegenerative diseases. The effective amount conversion between each animal is that those skilled in the art can easily convert based on the knowledge

For example, when being administered into a mouse, an effective amount of Nelumbo nucifera leaf water extract (NLWE) is at least approximately 10 g/kg; more preferably, an effective amount of Nelumbo nucifera leaf water extract (NLWE) is approximately from 1 g/kg to 100 g/kg. When being administered into a person, an effective amount of Nelumbo nucifera leaf water extract (NLWE) is at least approximately 1.1 g/kg; more preferably, an effective amount is approximately from 0.11 g/kg to 11 g/kg.

For example, when being administered into a mouse, an effective amount of Nelumbo nucifera leaf alcoholic extract (NLAE) is at least approximately 1 g/kg; more preferably, an effective amount of Nelumbo nucifera leaf alcoholic extract (NLAE) is approximately from 0.1 g/kg to 10 g/kg. When being administered into a person, an effective amount of Nelumbo nucifera leaf alcoholic extract (NLAE) is at least approximately 0.11 g/kg; more preferably, an effective amount is approximately from 0.01 g/kg to 1.1 g/kg.

In one aspect of the invention, wherein the subject is suffering from a degenerative neurological disease. In one embodiment, wherein the degenerative neurological disease is Alzheimer's disease. In another embodiment, wherein the degenerative neurological disease is Parkinson's disease.

In another aspect of the invention, wherein the subject is suffering from dementia. In one embodiment, wherein the dementia is caused by neurodegeneration. In another embodiment, wherein the dementia is vascular dementia.

In another aspect of the invention, wherein the Nelumbo nucifera leaf extract increase adult hippocampal neurogenesis by increasing DCX protein expression and activating BDNF/TrkB/CREB signaling pathway.

In another aspect of the invention, the main effective ingredients contained in each mg of Nelumbo nucifera leaf water extract (NLWE) are as follows: 6.11±0.15 μg gallic acid, 7.42±0.69 μg catechin, 3.55±0.04 μg peltatoside, 4.65±0.14 μg rutin, 2.83±50.1 μg isoquercitrin, 8.81±0.18 μg miquelianin, and 0.75±0.05 μg astragalin

In another aspect of the invention, the main effective ingredients contained in each mg of Nelumbo nucifera leaf alcoholic extract (NLAE) are as follows: 0.37±0.07 μg gallic acid, 24.8±1.84 μg catechin, 0.24±0.04 μg caffeic acid, 0.55±0.08 μg ρ-coumaric acid, 1.30±0.23 μg ferulic acid, 29.07±1.08 μg peltatoside, 8.89±0.34 μg rutin, 50.65±1.24 μg isoquercitrin, 89.05±2.47 μg miquelianin, 3.43±0.24 μg astragalin, 1.20±0.18 μg nuciferine, 6.70±0.18 μg quercitrin, 0.51±0.15 μg naringenin, and 4.7±0.14 μg hesperetin.

### EXAMPLES

Through the following specific embodiments, it can be further proved that the practical application scope of the present invention. It is only a preferred embodiment of the present invention, and does not limit the scope of the present invention. Therefore, any simple changes and modifications made in accordance with the scope of the present invention and the contents of the invention specification are still covered by the scope of the present invention.

Experimental Materials and Methods

Preparation of Nelumbo nucifera Leaf Extract

Preparation of Nelumbo nucifera Leaf Water Extract (NLWE)

The Nelumbo nucifera leaves were freeze-dried, grinded into powder at 4° C. with a grinder, and stored in a −20° C. freezer. Mix 100 g of dried Nelumbo nucifera leaf powder with 3000 mL of distillation-distillation H₂O (ddH₂O) and stir at 4° C. for one hour. After that, filter the coarse residue by suction filtration, collect the filtrate, and freeze drying to make lotus leaf water extract.

The main effective ingredients contained in each mg of Nelumbo nucifera leaf water extract (NLWE) are as follows: 6.11±0.15 μg gallic acid, 7.42±0.69 μg catechin, 3.55±0.04 μg peltatoside, 4.65±0.14 μg rutin, 2.83±50.1 μg isoquercitrin, 8.81±0.18 μg miquelianin, and 0.75±0.05 μg astragalin.

Preparation of Nelumbo nucifera Leaf Alcoholic Extract (NLAE)

Weigh 100 g of dried NLWE, and mix with 500 ml of methanol at 50° C. water bath for 3 hours. After incubation, collect the filtrate by filter suction. Repeatedly the extract procedures for 3 to 5 times. After completely extraction, concentrate the extract by vacuum concentration. The dried crude Nelumbo nucifera leaf alcoholic extract was reconstituted with 500 ml of ddH₂O, and further mixed with 200 ml of n-hexane to remove the pigment by placing in a separatory funnel. After standing overnight, the aqueous solution was collected and added 180 ml of ethyl acetate which is used to extract the ingredients in the water layer. Mix well and let stand overnight, collect the upper layer solution, and repeat the extraction procedures for 3 to 5 times. By using vacuum concentration method to remove the ethyl acetate, and finally dissolve in 20 ml ddH₂O and vacuum freeze-dried, the resulting powder was Nelumbo nucifera leaves alcoholic extract (NLAE) and stored in the refrigerator at −20° C. Before conducting cell experiments, the powder was dissolved in 50% alcohol solution and then sterile filtered, followed by high performance liquid chromatography (HPLC) analysis of NLAE. The results showed that the main effective ingredients contained in each mg of NLAE were as follows: 0.37±0.07 μg gallic acid, 24.8±1.84 μg catechin, 0.24±0.04 μg caffeic acid, 0.55±0.08 μg ρ-coumaric acid, 1.30±0.23 μg ferulic acid, 29.07±1.08 μg peltatoside 8.89±0.34 μg rutin, 50.65±1.24 μg isoquercitrin, 89.05±2.47 μg miquelianin 3.43±0.24 μg astragalin, 1.20±0.18 μg nuciferine, 6.70±0.18 μg quercitrin 0.51±0.15 μg naringenin, 4.7±0.14 μg hesperetin.

Animal Feeding and Grouping

Purchase a 5-week-old male C57BL/6 black mouse from the National Laboratory Animal Center. The weight of the mouse at the time of purchase was about 20 grams. It was kept in the Experimental Animal Center of Chung Shan Medical University. The life cycle is 12 hours of light and 12 hours of darkness, and the time of light is from 6 am to 6 pm. The temperature was maintained at room temperature 22±2° C., and the humidity was 60±5%. After 36 mice were raised to 16 weeks of age, they were randomly divided into control group or drug groups. The drug groups were including NLWE group (the treatment dosage was 10 g NLWE per kg of body weight) and NLAE group (the treatment dosage is 1 g NLAE per kg of weight). Animals in the control group were fed with general feed, while the drug groups mixed NLWE or NLAE into the general feed for mice. After 14-day treatment, the animals were euthanized with carbon dioxide in a high-pressure barrel, and blood was collected from the heart. Perfusion with PBS was used to wash away the whole body blood of the mouse. After the blood was washed, 6 mice from each group were taken for pre-fixation using 4% paraformaldehyde perfusion. After the fixation was completed, the mouse brain was removed. To facilitate follow-up tissue immunostaining and observe the distribution and expression of DCX, NeuN and PCNA in the dentate gyrus of the hippocampus. The other 6 mice in each group took fresh hippocampal tissue for Western blotting after PBS perfusion, and observed the protein expression of DCX, BDNF, TrkB and p-CREB.

Immunohistochemical Staining

Embed the mouse brain in paraffin, slice continuously at 3 μm, put it in 38° C. water after cutting, make it fully stretched, and attach it to the center of the coated slide (protein:glycerol is 1:1). Place the slides on a drying table at 38° C. to dry. Dewax with 100% xylene three times in an oven at 65-70° C. for 30 minutes, 5 minutes each time. After dewaxing, return water with high to low concentration alcohol, in order of 100%, 95%, 80%, 75% and ddH₂O, 5 minutes each. Place the slide in citric acid solution (0.01 M, pH=6) and heat to 100° C. in water for 20 minutes for antigen retrieval. After cooling, wash with 0.1% Tween in PBS solution (PBST) 3 times, then react with DAB (3,3′-Diaminobenzidine) color reagent inhibitor for 10 minutes. After the reaction was completed, add the primary antibody of the protein to be detected, act in a humid dark box at 37° C. for 1 hour, then wash with PBST 3 times, and then treat it with horseradish peroxidase multimer at room temperature for 20 minutes. Wash three times with PBST, add equal proportion of DAB coloring agent and DAB hydrogen peroxide (1:1) to make the tissue color for about 1 minute, and wash with ddH₂O for 5 minutes. After dyeing with hematoxylin as the background for 30 seconds, the excess dye has been washed with ddH₂O, and then dehydrated with low to high concentration alcohol (sequentially 70%, 80%, 95%, and 100% alcohol). After 5 minutes, finally immersed in xylene, and then sealed with mounting medium.

BDNF Quantitative Analysis

Enzyme-linked immunosorbent assay (ELISA) was used to detect the content of BDNF in serum by following the product manual of the purchased BDNF ELISA kit (ab212166, abcam). First, add 50 μL of mouse serum to the well plate, then add 50 μL of antibody cocktail, seal the plate on a shaker, and react at room temperature for 1 hour. 350 μL wash buffer, PT was washed three times. After washing, excess liquid was removed and 100 μL TMB (3,3′, 5,5′-tetramethylbenzidine) substrate was added and placed on the shaker in the incubator in the dark for 10 minutes, then add 100 μL stop solution, place on a shaker, react at room temperature for 1 minute, and measure the absorbance at 450 nm with an enzyme immunoassay analyzer. At the same time, the human BDNF standard was used to prepare the standard curve, and the content of BDNF in the mouse serum was calculated, and the result unit was expressed in μg/mL.

In this study, the cell model established by human neuroblastoma SH-SY5Y was used to observe the effect of lotus leaf extract in promoting nerve proliferation.

Cell Culture

SH-SY5Y cells were cultured in Dulbecco's modified Eagle medium-high glucose culture medium (DMEM-High glucose, pH 7.2-7.4) with 10% fetal bovine serum (FBS), 1.5 g/L sodium bicarbonate, 2 mM L-glutamine, 1 mM pyruvate Sodium, 0.1 mM non-essential amino acids solution and 1 mM antibiotic (Penicillin/Streptomycin/Amphotericine B, PSA) and placed in a cell incubator with a temperature of 37° C., a humidity of 95%, and 5% CO₂. Change the fresh medium every 2 to 3 days until the cells grow to reach 80 to 90% confluence and then subculture.

Subculture

When the cultures reached a confluence of 80˜90%, remove the cultured medium, wash with 5 mL phosphate buffer solution (PBS) 2 times, and then add 2 mL 0.5% trypsin-EDTA to incubate at 37° C. for a few minutes. After incubation, tap the flask to de-attach the cells from the flask, then add 3 mL of the culture solution to stop the reaction, and transfer the de-attached cells to the tube. After centrifugation at 1000 rpm for 5 minutes, remove the supernatant and add 2 ml of fresh culture medium to break up the cells evenly, take 1 mL of cells into a culture flask containing 9 ml of fresh culture medium and continue culturing.

Cell Survival Analysis

3-(4,5-Dimethylthiazol-2-yl)-2,5-diphenyltetrazolium bromide, MTT is a yellow dye that accepts hydrogen ions and acts on the respiratory chain in mitochondria. The principle of MTT is succinate dehydrogenase (SDH) and cytochrome C of the living cells in the mitochondria can convert the tetrazolium bromide of MTT to blue-purple formazan crystals and then deposited in cells. SDH in dead cells is no enzyme activity, and MTT cannot be metabolized and reduced. Therefore, by detecting the absorbance value of the ability of cells to convert yellow-colored MTT to blue/purple formazan crystals can be known to estimate the cell survival rate.

SH-SY5Y cells were cultured in 96-well dishes (1×10⁴ cells/well) for 24 hours, and then treated with different concentrations of NLAE (0, 25, 50, 100 μg/mL). After 24 hours of treatment, remove the cultured medium, add a culture solution containing 0.5 mg/mL MTT for 4-hour incubation. Remove the culture solution after 4 hours of reaction, add 200 μL isopropanol to dissolve the crystal, and read the absorbance at a wavelength of 563 nm by using an enzyme immunoassay analyzer to observe the cell survival rate.

Cell Proliferation Analysis

Bromodeoxyuridine (BrdU) is a synthetic nucleoside, which is an analog of thymidine. During DNA replication, it will compete with thymidine to bind to newly synthesized DNA. Further, the BrdU labeled on the DNA is detected by an antibody that can detect BrdU through immunochemical methods, so it can be used to detect the proliferation ability of cells. The BrdU cell proliferation analysis kit used in this research was purchased from Merck Millipore QIA58 and the following experiments were performed according to the user manuals.

SH-SY5Y cells were incubated in a 96-well dish (1×104 cells/well) for 24 hours, and then simultaneously add 180 μL of different concentrations of NLAE (0, 25, 50, 100 μg/mL) and 20 μL BrdU for 24 hours. After 24 hours of incubation, remove the supernatant and add 200 μl Fixative/Denaturing solution. After standing at 25° C. for 30 minutes, remove the contents and add 100 μL anti-BrdU antibody (according to 1:2000 ratio for dilution), let stand at 25° C. for 1 hour, remove the contents, wash with wash buffer 3 times and add 200 μL peroxidase goat anti-mouse IgG (diluted at 1:2000 ratio) at 25° C. for 30 minutes. After that, remove the contents, wash with wash buffer 3 times, then rinse with ddH₂O. After washing, add 100 μL substrate solution, and keep at 25° C. in the dark for 15 minutes, and then add 100 μL of stop solution to stop the reaction. Use an enzyme immunoassay analyzer to read the absorbance at 450 nm in 30 minutes to observe the cell proliferation.

Western Blotting

Mouse hippocampal gyrus protein extraction: Take the hippocampus from the left and right sides of the mouse and add 150 μL of protein lysate (RIPA buffer is used as protein lysate in this study, which includes 50 mM NaCl, 0.5% Deoxycholic acid, 50 mM Tris-Base, 1% NP-40, 1% SDS, 10 μg/mL PMSF, 10 μg/mL Leupeptin, 1% Protease inhibitor, Phosphatase inhibitor, pH7.5) into a glass test tube, grind on ice with a grinder for 5 minutes, and then centrifuge at 12,000 rpm for 20 minutes at 4° C., draw the supernatant into a new tube and store at −20° C. This is the mouse hippocampal protein.

Cell protein extraction: SH-SY5Y cells were cultured in 75T culture flasks (2.37×10⁶ cells/vial) for 24 hours, then treated with different concentrations of NLAE (0, 25, 50, 100 μg/mL) for 24 hours. After 24-hour incubation, wash twice with PBS, add 2 mL of trypsin-EDTA to de-attach cells. Further, add 3 mL of culture medium containing 10% FBS was to neutralize the enzymatic activity of trypsin, and then centrifuged at 1000 rpm for 5 minutes to get the cell pellet. The pellet was washed with PBS and added 100 μL of protein lysis buffer (RIPA buffer), grinded on ice for 3 minutes, and then centrifuged at 12,000 rpm for 20 minute. Take the supernatant and store at −20° C. until analysis.

The protein extracts of hippocampal tissues or cells of each group were subjected to protein quantification, and each group was diluted to the same concentration according to the result of the quantification, added 5 times sample buffer and mixed uniformly, and heated at 100° C. for 10 minutes. The protein sample to be analyzed is subjected to protein electrophoresis, and then the protein on the gel was transferred to the nitrocellulose membrane through protein transfer. The nitrocellulose membrane was reacted with 5% skim milk at room temperature for 1 hour to block non-specific antigen binding; after that, a primary antibody was added to act at 4° C. overnight, and then washed 3 times with PBST (5 minutes/time), added the secondary antibody for 1 hour at room temperature, washed with PBST 3 times (5 minutes/time), added HRP coloring agent and detected the signal under the illuminometer.

Statistical Analysis

The test results of various indicators of animal experiments were first analyzed using Sigma plot 10.0 statistical software. One-way ANOVA was used for analysis, and Duncan's Multiple Range Test was used to compare whether there was a statistically significant difference between the control group and the drug group (p<0.05). The results of the cell experiment were compared with the differences between the groups by Student's t test. When p<0.05, it indicated that there were statistically significant differences.

Experimental Results

Effects of NLWE and NLAE on Neurogenesis of SGZ in Hippocampal Dentate Gyrus of Mice

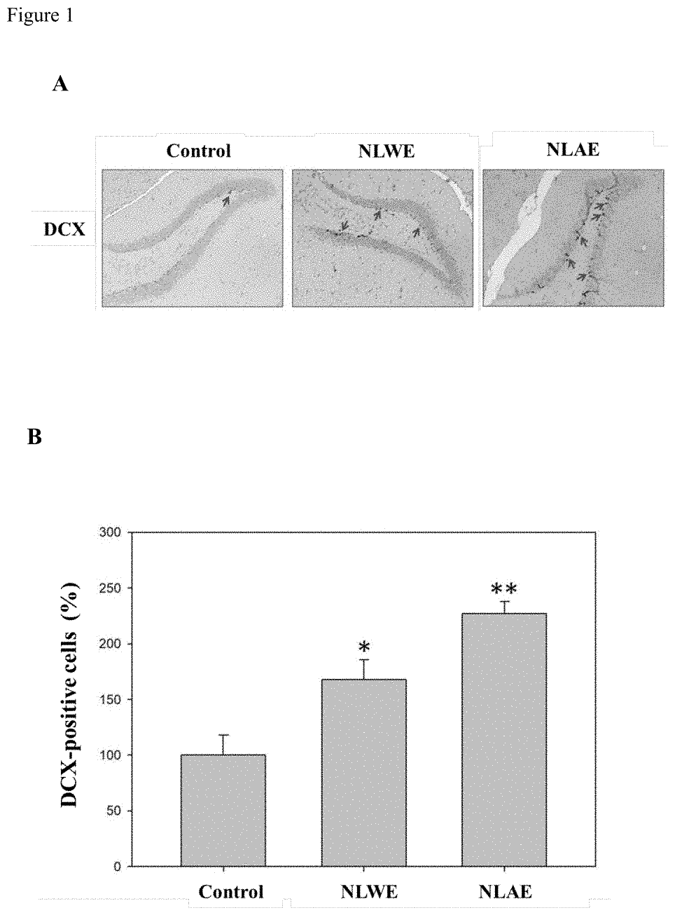

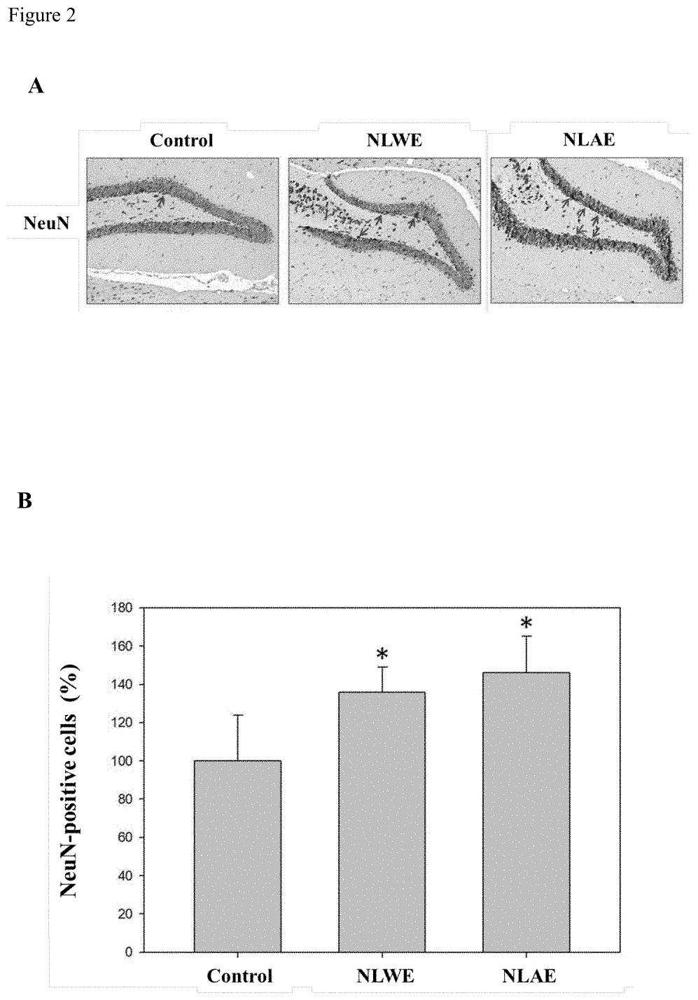

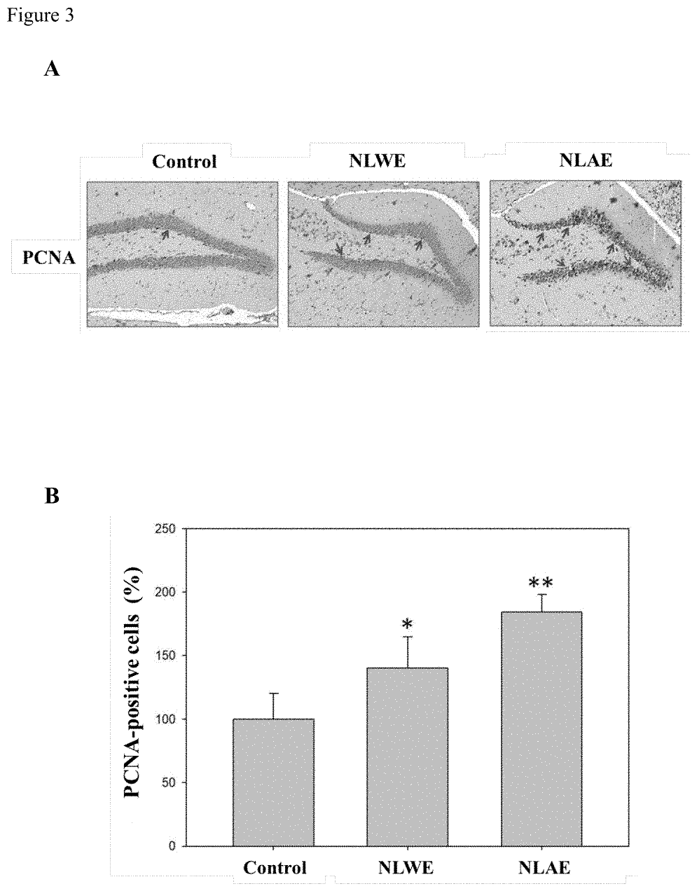

To observe whether Nelumbo nucifera leaf extract can promote SGZ nerve regeneration in the mouse dentate gyrus, after the mouse sacrifice, take its hippocampus and use immunochemical tissue staining to observe the immature neuron differentiation indicator protein, DCX, the index protein of nucleus of mature neurons, NeuN and cell proliferation index protein, PCNA. The results show that there is no DCX and PCNA expression, but only a small amount of NeuN expression in the hippocampal dentate SGZ of the control group mice. However, there is a significantly increase of DCX, NeuN and PCNA expression in the hippocampal dentate SGZ of mice fed with Nelumbo nucifera leaf extract. Especially, the increased expression is more significantly in mice fed with NLAE. From the above results, it is known that both NLWE and NLAE can effectively promote the neural regeneration of SGZ in the hippocampal dentate gyrus of C57BL/6 mice (FIG. 1-3).

The Effect of NLWE and NLAE on the Serum BDNF Content in Mice

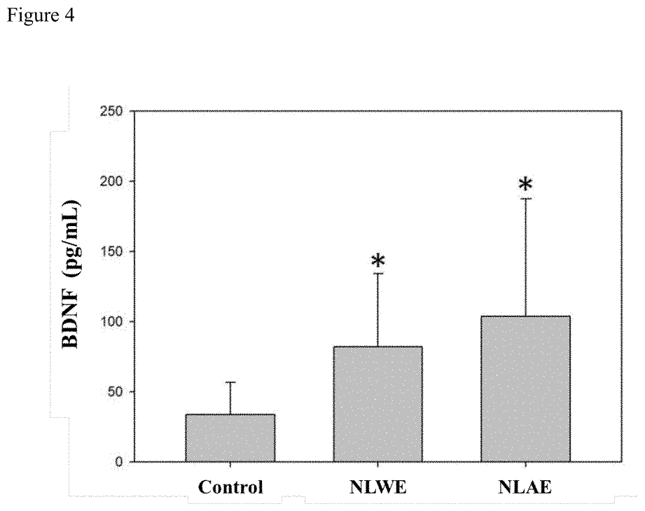

BDNF is the main neurotrophic factor in the brain, mainly expressed in the hippocampus, cerebellum and cerebral cortex, and plays an important role in the formation of long-term memory. Previously studies have shown that BDNF can promote the formation of synapses in brain nerve cells, increase synaptic plasticity, promote the proliferation and differentiation of neural stem cells, and resist the damage of harmful factors such as oxidative inflammation to the brain. Therefore, this invention intends to further explore whether NLWE and NLAE can increase the secretion of BDNF to promote neurite regeneration in the hippocampus. The results show that compared with the control group, the BDNF content in the serum of mice fed NLWE or NLAE increased significantly (p<0.05). In addition, the increase in the NLAE group is more significant than in the NLWE group. From the results, we can know that NLWE and NLAE can effectively increase the secretion of BDNF in serum (FIG. 4).

Effects of NLWE and NLAE on Neural Regeneration-Related Proteins in the Hippocampal Gyrus of Mice

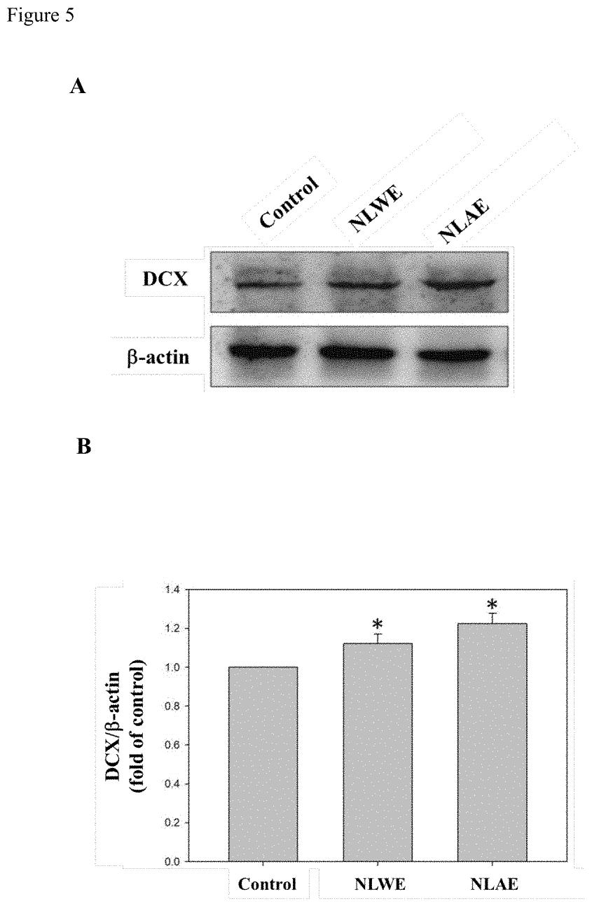

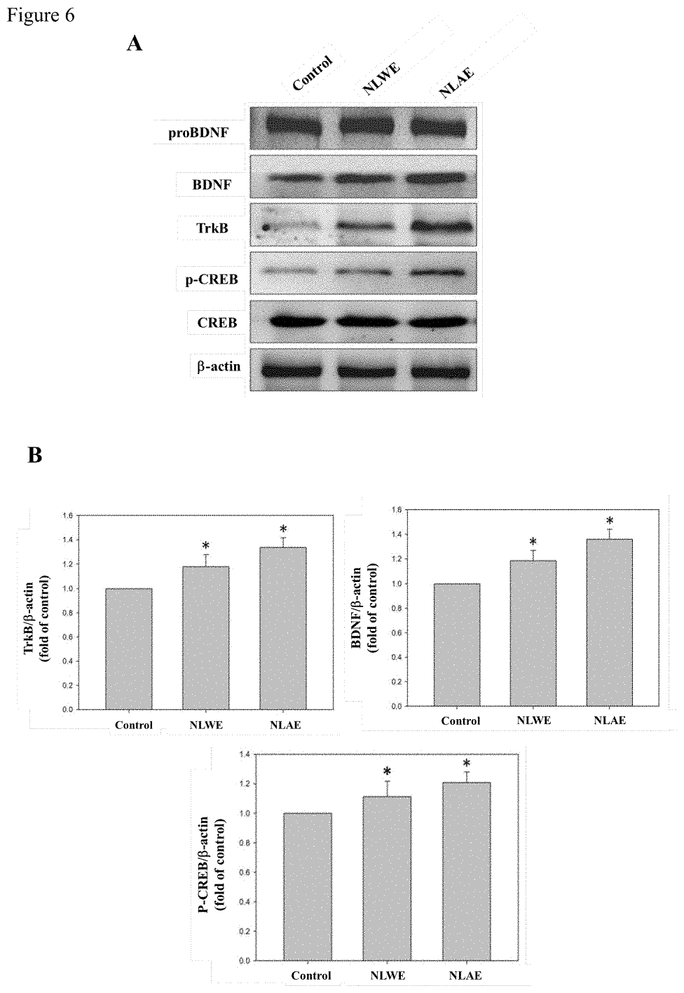

Previous studies have shown that DCX is an important indicator of immature neuronal differentiation, and the BDNF/TrkB/CREB signaling pathway can regulate nerve growth, differentiation, and remodeling. In order to verified the mechanism of NLWE or NLAE in promoting neural regeneration, the expression of neuronal regeneration-related proteins in the hippocampus of mice were evaluated by Western blotting. The results show that the protein expression of DCX, BDNF, TrkB, p-CREB in the hippocampus of mice fed the NLWE and NLAE groups are higher than that of the control group, especially the results of the NLAE group are more significant (FIG. 5-6). Therefore, according to the above results, it is known that NLWE and NLAE can increase the expression of DCX protein and activate the BDNF/TrkB/CREB signaling pathway to promote neurite regeneration in mouse hippocampus.

Toxicity Test of NLAE in Human Neuroblastoma SH-SY5Y Cells

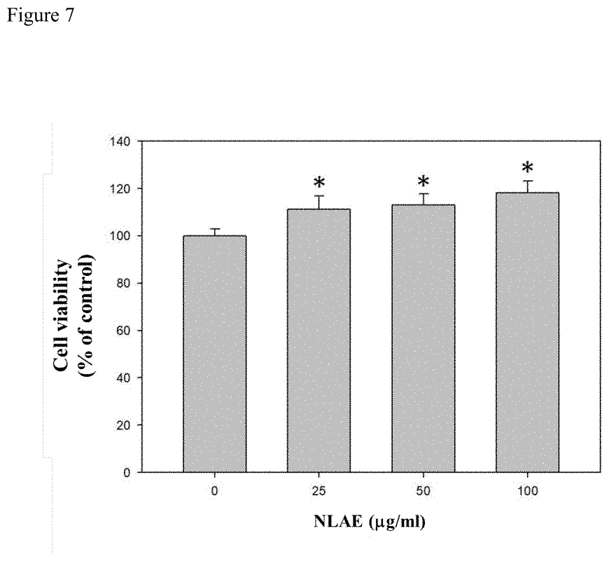

In order to ensure that the dose of NLAE used would not cause the death of SH-SY5Y cells, in this experiment, SH-SY5Y cells were seeded in 96-well plate (1×10⁴ cells/well), after 24 hours of culture, add NLAE of different concentrations (0, 25, 50, 100 μg/mL) for 24 hours, followed by MTT assay for cell viability. The results show that high concentration of NLAE (100 μg/mL) does not cause a decrease in the survival rate of SH-SY5Y cells. However, compared with the control group, the cell survival rate of the NLAE-treated group has a significantly increased trend, and increases as the drug concentration increases. It can be known from the above results that the dose of NLAE selected in this experiment does not cause cell death, but tend to promote cell proliferation (FIG. 7).

The Proliferation Test of NLAE in Human Neuroblastoma SH-SY5Y Cells

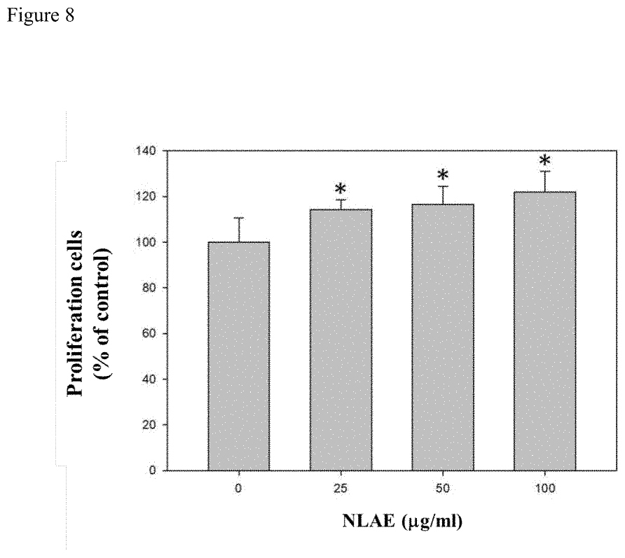

Next, to further evaluate the effects of NLAE on promoting the proliferation of SH-SY5Y cells, cells were grown in 96-well plate (1×10⁴ cells/well) for 24 hours, and then added with different concentrations of NLAE (0, 25, 50, 100 μg/ml) and BrdU for 24 hours. Detect the cell proliferation ability by BrdU cell proliferation kit. The results show that the proliferation ability significantly increases in NLAE treatment, which is a dose-dependent manner (FIG. 8).

Effect of NLAE on the Promotion of SH-SY5Y Neural Regeneration Related Protein

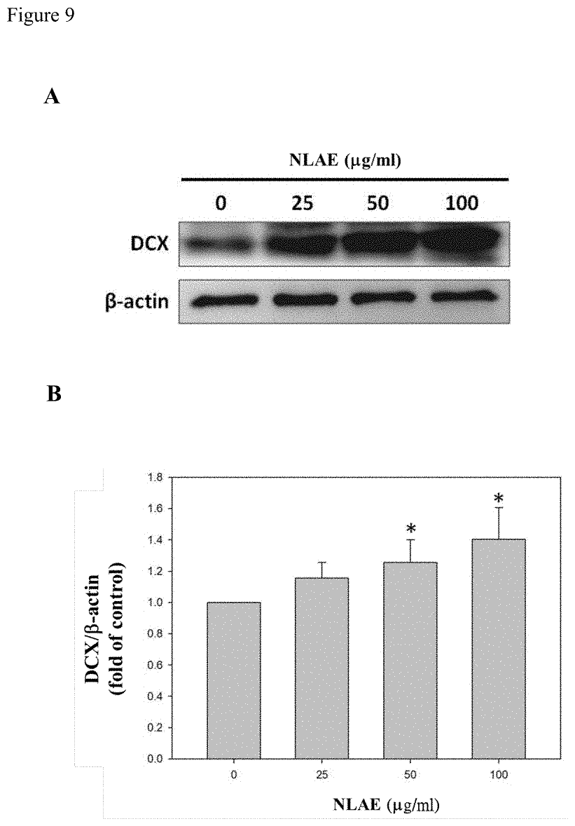

From the above results, it is known that NLAE effectively promotes the proliferation of SH-SY5Y cells, so further analysis of the expression of neuronal neonatal-related proteins to confirm the mechanism of promoting proliferation. Cells were cultured in 75T flask (2.37×10⁶ cells/vial) for 24 hours, treated with different concentrations of NLAE (0, 25, 50, 100 μg/ml) for 24 hours, and then observed the expression of neuronal neonatal associated protein by Western blotting. The results show that after treatment of NLAE with SH-SY5Y, the expression of immature neuronal differentiation index protein DCX is higher than that of the control group and the increase is in a dose-dependent manner. As a result, NLAE can promote SH-SY5Y neural regeneration by increasing the performance of DCX (FIG. 9).

What is claimed is:

1. A method of increasing adult hippocampal neurogenesis in a subject, comprising administering a composition comprising an effective amount of Nelumbo nucifera leaf extract to the subject, wherein the Nelumbo nucifera leaf extract is Nelumbo nucifera leaf water extract (NLWE), or Nelumbo nucifera leaf alcoholic extract (NLAE).

2. The method according to claim 1, wherein the Nelumbo nucifera leaf extract increase adult hippocampal neurogenesis by activating BDNF/TrkB/CREB signaling pathway.

3. The method according to claim 1, wherein the composition is a pharmaceutical composition or a food composition.

4. The method according to claim 1, wherein the subject is suffering from a degenerative neurological disease.

5. The method according to claim 4, wherein the degenerative neurological disease is Alzheimer's disease.

6. The method according to claim 4, wherein the degenerative neurological disease is Parkinson's disease.

7. The method according to claim 1, wherein the subject is suffering from dementia.

8. The method according to claim 6, wherein the dementia is caused by neurodegeneration.

9. The method according to claim 6, wherein the dementia is vascular dementia.

10. The method according to claim 1, wherein the subject is human.

11. The method according to claim 10, wherein the effective amount of Nelumbo nucifera leaf water extract is about 0.11-11 g/kg.

12. The method according to claim 10, wherein the effective amount of Nelumbo nucifera leaf alcoholic extract is about 0.011-1.1 g/kg.
